(19) United States
(12) Patent Application Publication
(10) Pub. No.: US 2013/0064861 A1
(43) Pub. Date: Mar. 14, 2013

(54) COMPOSITIONS AND METHODS FOR MODIFYING IN VIVO CALCIFICATION OF HYDROGELS

(71) Applicants: Zvi Schwartz, Atlanta, GA (US); Christopher S.D. Lee, Atlanta, GA (US); Hunter R. Moyer, Atlanta, GA (US); Barbara D. Boyan, Atlanta, GA (US)

(72) Inventors: Zvi Schwartz, Atlanta, GA (US); Christopher S.D. Lee, Atlanta, GA (US); Hunter R. Moyer, Atlanta, GA (US); Barbara D. Boyan, Atlanta, GA (US)

(21) Appl. No.: 13/579,632

(22) PCT Filed: Feb. 17, 2011
(86) PCT No.: PCT/US11/25268

§ 371 Date: Nov. 5, 2012

Related U.S. Application Data
(60) Provisional application No. 61/305,296, filed on Feb. 17, 2010.

(51) Int. Cl. A61K 47/36; A61K 9/14

(52) U.S. Cl. 424/400; 514/779

(57) ABSTRACT

Provided herein according to some embodiments of the invention are methods of inhibiting or preventing calcification of hydrogels. Such methods may include combining the hydrogel with a buffer solution having a pH lower than 7.4; forming a hydrogel by crosslinking alginate in a solution comprising a bisphosphonate compound; and/or forming a hydrogel by crosslinking a polyanionic polymer with a polyvalent cation that is not Ca²⁺. Compositions that may be used in such methods are also provided herein. Also provided herein according to some embodiments of the invention are methods of bone regeneration and/or formation that include administering a hydrogel that does not encapsulate biological material that affects calcification and/or bone formation to an area of a subject's body that is in need of bone formation and/or regeneration.

### CROSS-REFERENCE TO RELATED APPLICATIONS

This application claims priority from U.S. Provisional Application Ser. No. 61/305,296, filed on Feb. 17, 2010, the disclosure of which is hereby incorporated by reference herein in its entirety.

### STATEMENT REGARDING FEDERALLY SPONSORED RESEARCH OR DEVELOPMENT

This invention was made with United States Government support under Grant No. W81XWH-08-1-0704, awarded by the U.S. Department of Defense. The United States Government may have certain rights in the invention.

### FIELD OF THE INVENTION

The present invention relates to hydrogels. More particularly, the present invention relates to compositions and methods of using hydrogels in biomedical applications.

### BACKGROUND OF THE INVENTION

Alginate hydrogels have been used for a wide variety of tissue engineering and regenerative medicine application due to their many desirable properties. For example, alginate hydrogels may have favorable mass transfer properties, may be molded into specific shapes, may have adjustable degradation kinetics, may support a range of different cell phenotypes, may be mechanically and biochemically modified, may support cell differentiation in large animal models, and may be biocompatible for delivery of cells in human trials. The most common method of incorporating bioactive molecules or cells into alginate matrices is via extrusion, in which an alginate suspension is extruded through a needle to form droplets that fall into a solution that contains polyvalent cations causing alginate crosslinking. Alginate microspheres can also be created by using air flow or high electrostatic potentials to overcome surface tension, and have been used to encapsulate and deliver pancreatic islets. In addition to cell delivery, hydrogel microspheres have also been used for spheroid cell culture, drug delivery, and as injectable tissue fillers.

Various pre-clinical and clinical studies have reported alginate calcification, which presents a critical challenge in developing large scale applications using this hydrogel. Alginate calcification in vivo affects mass transfer in and out of the hydrogel and may prevent reabsorption and create unwanted mineralization foci within the tissue. Therefore, methods to prevent and control alginate calcification would be desirable.

### SUMMARY OF THE INVENTION

Provided according to some embodiments of the invention are methods of inhibiting or preventing calcification of a hydrogel in vivo that include combining the hydrogel with a non-phosphate buffer solution having a pH of less than 7.4; and administering the hydrogel to a subject, wherein the hydrogel is formed by crosslinking a polyanionic polymer with a polycation. In some embodiments, combining the hydrogel with the non-phosphate buffer solution includes crosslinking the polyanionic polymer with the polyvalent cation in the non-phosphate buffer solution to form the hydrogel.

Also provided according to embodiments of the invention are methods of inhibiting or preventing calcification of hydrogel in vivo that include forming hydrogel by crosslinking a polyanionic polymer with a polycation in a solution comprising a bisphosphonate compound; and administering the formed hydrogel to a subject.

Additionally provided are methods of inhibiting or preventing calcification of hydrogel in vivo that include forming hydrogel by crosslinking a polyanionic polymer with a polycation in a solution comprising a bisphosphonate compound; and administering the formed hydrogel to a subject.

Further provided according to embodiments of the invention are methods of inhibiting or preventing calcification of hydrogel in vivo that include forming hydrogel by crosslinking a polyanionic polymer with a polyvalent cation that is not Ca²⁺; and administering the formed hydrogels to a subject.

In some embodiment, the polyanionic polymer includes a polyanionic polysaccharide, and in some embodiment, the polyanionic polymer is alginate. In some embodiments, the hydrogel is administered as particles having a diameter in a range of 30 μm to 2 mm. In some embodiments, the hydrogel particles have a diameter in a range of 175 μm to 350 μm.

In some embodiments of the invention, the hydrogel encapsulates biological material, and in some cases, the biological material is encapsulated during crosslinking of the hydrogel.

In some embodiments of the invention, administering the hydrogel includes injecting and/or implanting the hydrogel into the subject. In some cases, the hydrogel does not calcify in the subject within 8 weeks.

Also provided herein are hydrogel compositions. In some embodiments, hydrogel compositions include hydrogel formed by crosslinking a polyanionic polymer and a polycation; and a bisphosphonate compound. In some embodiments, hydrogel compositions include hydrogel formed by crosslinking a polyanionic polymer and a polycation; and a non-phosphate buffer solution.

### BRIEF DESCRIPTION OF THE FIGURES

The foregoing and other objects, features and advantages of the invention will become more apparent from the following more particular description of exemplary embodiments of the invention and the accompanying drawings. The drawings are not necessarily to scale, emphasis instead being introduced upon illustrating the principles of the invention.

FIG. 1A shows the phosphate concentration of a buffered bath over time for different amounts of alginate added to 25 mls of 4 mM (NH₄)₂HPO₄ in 0.05 M Tris buffer at pH 7.4. FIGS. 1B-1D show the FTIR spectra of (B) lyophilized alginate beads, (C) precipitate from the bath, and (D) pure hydroxyapatite.

FIGS. 2A-2D provide gross-visualization of alginate microbead mineralization. (A) Microbeads before implantation or injection under a light microscope. (B) Visualization of mineralized microbeads 3 months post-implantation under a light microscope. (C) Mineralized microbeads 3 months post-implantation. (D) Mineralized microbeads 1 month post-injection. Bar represents 100 μm for all images.

FIGS. 3A-3D provide histology of in vivo microbeads. von Kossa with nuclear fast red counter stain were used to determine calcification for representative (A) non-buffered, (B) barium chloride, (C), bisphosponate, (D) buffered samples in vivo after 2 months. Bar represents 100 μm for all images.

FIGS. 4A-4D provide MicroCT analysis of non-buffered in vivo samples. (A) Representative X-ray cross-section of subcutaneously implanted non-buffered microbeads after 5 weeks in vivo. (B) 3-D reconstruction of subcutaneously implanted non-buffered microbeads after 5 weeks in vivo. (C) Representative sagittal X-ray cross-section of intramuscularly implanted non-buffered microbeads near the tibia after 5 weeks in vivo. (D) 3-D reconstruction of intramuscular implanted non-buffered microbeads along with the tibia after 5 weeks in vivo. Bar represents 1 mm for all images.

FIGS. 5A-5D provide FTIR spectra of (A) non-buffered microbeads after 5 weeks in vivo, (B) buffered microbeads after 5 weeks in vivo, (C) HEPES powder used to buffer the crosslinking solution, and (D) alginate powder used to make the microbeads.

FIG. 6 provides XRD Spectra of HEPES powder used to buffer the crosslinking solution, alginate powder used to make the microbeads, buffered microbeads after 5 weeks in vivo, NaCl from the database, non-buffered microbeads after 5 weeks in vivo, and hydroxyapatite from the database.

FIGS. 7A and 7B are SEM images of (A) Lyophilized non-buffered microbeads after 5 weeks in vivo and (B) Lyophilized buffered microbeads after 5 weeks in vivo.

### DETAILED DESCRIPTION OF EMBODIMENTS OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

All patents, patent applications and publications referred to herein are incorporated by reference in their entirety. In the event of conflicting terminology, the present specification is controlling.

The embodiments described in one aspect of the present invention are not limited to the aspect described. The embodiments may also be applied to a different aspect of the invention as long as the embodiments do not prevent these aspects of the invention from operating for its intended purpose.

Provided herein are methods of modulating the calcification in vivo of hydrogels formed from polyanionic polymers that are crosslinked with polycations. In some embodiments, the methods described herein inhibit or prevent calcification. Such methods include combining hydrogel with a non-phosphate buffer solution having a pH lower than 7.4; forming hydrogel by crosslinking a polyanionic polymer with a polycation in a solution comprising a bisphosphonate compound; and/or forming hydrogel by crosslinking a polyanionic polymer with a polyvalent cation that is not Ca²⁺. Also provided are methods of bone regeneration and/or formation that include administering hydrogel that does not encapsulate biological material that affects calcification and/or bone regeneration to an area of a subject's body that is in need of bone formation and/or regeneration.

As used herein, the term “solution” may refer to homogeneous solutions, but also dispersions, colloids, emulsions, and the like.

### Hydrogels

The hydrogels described herein are formed by crosslinking a polyanionic polymer with a polycation. Any suitable polyanionic polymer may be used to form the hydrogels. In some embodiments, the hydrogels described herein are formed with polyanionic polysaccharides. In some embodiments, the polyanionic polysaccharides include alginic acid and/or salts thereof. In some embodiments, the polyanionic polymer may be present prior to hydrogel formation as a salt such as a metal salt, such as sodium, potassium and the like. Any suitable molecular weight may be used, however, in some embodiments, the molecular weight of the polyanionic polymer is 50,000 to 250,000 g/mole. Furthermore, any suitable combination of polyanionic polymers may be used. As such, when the term “a polyanionic polymer” is used, this may refer to one polyanionic polymer or to two or more different polyanionic polymers.

As used herein, alginic acid and its salts, which are also referred to herein as alginate, includes synthetic and naturally occurring anionic polysaccharides that include 1,4-linked-β-D-mannuronic acid and α-L-guluronic acid in any suitable proportion. In some embodiments, the alginates vary from 70% mannuronic acid and 30% guluronic acid to 30% mannuronic acid and 70% guluronic acid. The alginate may be in any suitable form, and any suitable molecular weight, including linear copolymer with homopolymeric blocks of (1-4)-linked β-D-mannuronate (M) and its C-5 epimer α-L-guluronate (G) residues, respectively, covalently linked together in different sequences or blocks. Additionally, in some embodiments, the monomers can appear in homopolymeric blocks of consecutive G-residues (G-blocks), consecutive M-residues (M-blocks), alternating M and G-residues (MG-blocks), or randomly organized blocks. In particular embodiments, MVG and/or LVM alginate is used to form alginate hydrogels. In some embodiments, the alginate is a metal salt, such as sodium alginate. Any suitable molecule weight may be used, however, in some embodiments, the molecular weight of the alginate is 50,000 to 250,000 g/mole.

Hydrogels may be formed by the crosslinking of the polyanionic polymer with a polycation. Bound polycations can be obtained from various commercial, natural or synthetic sources that are well known in the art. In particular, cationic metal ions can include but are not limited to aluminum, barium, calcium, iron, manganese magnesium, strontium and zinc. In some embodiments, the metal ions are calcium and zinc or the salts thereof, such zinc acetate, calcium acetate or chloride salts. Water soluble small molecules and salts can also be used such as ammonium sulfate, acetone, ethanol and glycerol. In some embodiments, the polycation is in the +2 oxidation state. Furthermore, any suitable combination of polycations may be used.

The hydrogels may be present in any suitable physical form. However, in some embodiments, the hydrogel may be present in particulate form. In particular embodiments, the diameter of the hydrogel particles is in a range of 30 μm to 2 mm. For non-spherical particles, the diameter is considered to be the largest distance across the particle. In particular embodiments, the hydrogel particles have a diameter in a range of 175 μm to 350 μm.

In some embodiments of the invention, the hydrogels may be used to encapsulate biological material, including, but not limited to, microorganism, cells, cell products, or biological molecules. Biological molecules are molecules that are produced by a living organism, and this also refers to synthetic analogs of such molecules. Examples of biological molecules include carbohydrates such as glucose, disaccharides and polysaccharides; proteins, including growth factors and cytokines, lipids (including lipid bilayers); and nucleic acids, such as DNA and RNA. Biological molecules may also be small molecules, including monomers and oligomers of other biological molecules, e.g., nucleic acids, nucleotides, fatty acids, etc. The biological molecules may be naturally occurring or synthetic, or may include both naturally occurring and synthetic portions. Two or more biological materials may also be encapsulated together in a hydrogel described herein.

Any suitable type of cell may be encapsulated in the hydrogels, including but not limited to neural cells (including cells of the peripheral and central nervous systems, in particular, brain cells such as neurons, oligodendricytes, glial cells, astrocytes), lung cells, cells of the eye (including retinal cells, retinal pigment epithelium, and corneal cells), epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., mesenchymal stem cell, neural stem cell, liver stem cell, adipose stem cell). As still a further alternative, the cell may be a cancer or tumor cell. Moreover, the cells can be from any species of origin.

Any suitable method of forming hydrogels may be used. In some embodiments, the hydrogels are formed by a method described herein. In some embodiments, the polyanionic polymer is UV light sterilized and dissolved in a water or saline solution. In some embodiments, the saline solution has a salt concentration in a range of 0 to 165 mM. The polyanionic polymer may then be seeded with biological material such as cells. The seeded solution may then be added to a crosslinking solution that includes a polyvalent cation and, optionally, other additives, such as those that may alter the ionic strength of the solution. In some embodiments, the ionic strength of the crosslinking solution is modified so that it is isotonic with biological material to be encapsulated in the hydrogel. In some embodiments, glucose may be included in the crosslinking solution to alter the ionic strength. Hydrogels made in non-buffered crosslinking solutions may be washed and stored in sodium chloride (saline).

### Methods of Inhibiting or Preventing Calcification of Hydrogel In Vivo

Provided according to embodiments of the invention are methods of inhibiting or preventing calcification of hydrogel in vivo. As used herein, the term “inhibition of calcification” means that the calcification is reduced using a method described below relative to a hydrogel formation process wherein calcium is used to crosslink the alginate, the solution is at or above physiological pH (7.4) and no bisphosphonate is present in the crosslinking solution. The term “prevention of calcification” refers to no calcification being detected via undecalcified histology and X-ray detection methods after 8 weeks in vivo.

### Buffer Solutions

In some embodiments, the hydrogels may be combined with a non-phosphate buffer solution having a pH lower than 7.4. In some embodiments, the hydrogels thus formed may then be administered to a subject. Combining the hydrogels with the non-phosphate buffer solution may be performed after the hydrogels are formed, or the hydrogels may be crosslinked in the non-phosphate buffer solution. In some cases, the alginate may be crosslinked in one buffer and the stored or introduced to one or more additional buffer solutions. In some embodiments, the hydrogels are formed by the general procedure described above with respect to hydrogels, but with a non-phosphate buffer included in the crosslinking solution such that the crosslinking solution has a pH of less than 7.4.

Any suitable buffer solution may be used provided that the pH is less than 7.4 and it does not have significant phosphate concentration. Examples of buffer solutions that may be used in embodiments of the invention include 4-(2-hydroxyethyl)-1-piperazineethanesulfonic acid (HEPES) and hydroxymethyl)aminomethane (Tris). Combination of buffering compounds may also be used. In particular embodiments, the pH is in a range of 7.3 to 7.4. In some embodiments, the pH is less than 7.3, and in particular embodiments, the pH is in a range of 6.4 to 7.3.

The buffered hydrogel may then be washed and/or stored in a basal medium. An example of a basal medium is Dulbecco's modified eagle medium (DMEM) [Invitrogen, Carlsbad, Calif., USA]. The cells may also be stored at temperatures suitable for cells, such as 37° C.

Also provided according to embodiments of the invention are compositions that include an hydrogel described herein and a non-phosphate buffer solution described herein.

### Bisphosphonate Solutions

In some embodiments of the invention, methods of inhibiting or preventing calcification of hydrogel in vivo include forming hydrogel by crosslinking a polyanionic polymer with a polycation in a solution comprising a bisphosphonate compound. In some embodiments, the hydrogel thus formed may be administered to a subject. Any suitable bisphosphonate compound may be used. In some embodiments, the bisphosphonate includes aledronic acid and/or a salt thereof (e.g., aledronate). Other examples include pamidronate, neridronate, olpadronate, ibandronate, risedronate, zoledronate, etidronate, clodronate and tiludronate. Any suitable combination of bisphosphonates may also be used. In some embodiments, the bisphosphonate compound is present in the solution at a concentration in a range of 1 μM to 1 mM. In some embodiments, the hydrogels are formed by the general procedure described above, but with a bisphosphonate compound included in the crosslinking solution.

Also provided according to embodiments of the invention are compositions that include an hydrogel and a bisphosphonate compound. In some embodiments, the bisphosphonate compound includes aledronic acid and/or salts thereof. Furthermore, in some embodiments, the bisphosphonate and hydrogel are present in solution and the bisphosphonate is present at a concentration in a range of 1 μM to 1 mM. The compositions may further include other pharmaceutically acceptable carriers, solvents, excipients, and the like, provided they do not significantly deleteriously affect the activity of the bisphosphonate.

Crosslinking without Calcium

In some embodiments of the invention, methods of inhibiting or preventing calcification of hydrogel in vivo include forming an hydrogel by crosslinking alginate using a polyvalent cation that is not Ca²⁺. In some embodiments, the hydrogels thus formed may be administered to a subject. Any suitable non-calcium polyvalent cation may be used. Examples include Ba²⁺, Mg²⁺ and Sr²⁺.

In some embodiments, the hydrogels are formed by the general procedure described above, but without using calcium as the polyvalent cation.

Methods of Bone Regeneration and/or Formation

Also provided according to embodiments of the invention are methods of bone regeneration and/or formation. Such methods may include administering hydrogel to an area of a subject's body that is in need of bone formation and/or regeneration; and allowing the hydrogel to calcify in the area of the subject's body. In some embodiments, the administered hydrogel does not encapsulate biological material that affects calcification and/or bone formation. This means that the hydrogels in these embodiments are not used to encapsulate biological material for bone formation or regeneration, but instead, the hydrogel itself is used for bone regeneration and/or formation. Some small amount of biological material may be associated with the hydrogels provided it does not significantly affect the calcification of the hydrogels. A biological material “significantly affects” the calcification if a change in calcification can be detected by undecalcified histology or X-ray imaging upon inclusion of the biological molecule.

In particular, the structure of polyanionic polymers such as alginate may facilitate controlled calcification for bone tissue engineering. Injectable, crosslinked-polymers that can then mineralize in situ without the presence of other biological or chemical factors may present an advantage over pre-mineralized scaffolds or bone morphogenetic proteins in that it avoids adverse immune responses, limits systemic side effects, and is minimally-invasive for simple orthopedic and reconstructive applications.

### Administering the Hydrogel to a Subject

As used herein, the term “administering” to a subject refers to any method of introducing the hydrogel into or onto the subject, including injecting and/or implanting the hydrogel. Other application methods such as topical application, transdermal patches and the like, may also be used to administer the hydrogel. As such, the methods may include introducing the hydrogel while it is dispersed in an aqueous solution. The hydrogel may also be administered in a pharmaceutical composition that may include other pharmaceutically acceptable carriers, solvents, excipients, and the like, provided they do not significantly deleteriously affect the activity of the remaining components. Combinations of hydrogels may also be administered concurrently or sequentially. Furthermore, in some embodiments, the hydrogels described herein may be used in combination with other therapeutic agents or regimens. The administration of hydrogel described herein may be prior, concurrent with or after the administration of other therapeutic agents.

The hydrogels described herein may be administered to any suitable subject. Subjects suitable to be treated with methods and compositions according to an embodiment of the invention include, but are not limited to, avian and mammalian subjects. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention. Illustrative avians according to the present invention include chickens, ducks, turkeys, geese, quail, pheasant, ratites (e.g., ostrich) and domesticated birds (e.g., parrots and canaries), and birds in ovo. The invention can also be carried out on animal subjects, particularly mammalian subjects such as mice, rats, dogs, cats, livestock and horses for veterinary purposes, and for drug screening and drug development purposes.

### EXAMPLES

### Example

### In Vitro Phosphorus Content

An in vitro study was designed to assess the ability of calcium-crosslinked alginate to sequester phosphate. Low viscosity sodium alginate [Kelco Corp., Chicago, Ill., USA] dissolved in 155 mM sodium chloride at a concentration of 12 mg/ml was dropped gently through a 25 gauge needle at a rate of 2-3 drops per second into a 102 mM calcium chloride bath. After 10 minutes, the beads (15-20 beads/10 ml alginate) were washed four times with 155 mM NaCl. The beads were suspended in 25 ml 4 mM (NH₄)₂HPO₄ in 0.05 M Tris buffer at pH 7.4, and removed at different time points over a 4 hour period (0, 0.5, 1, 2, and 4 hrs). After incubation at room temperature, the beads were collected by centrifugation, and the supernatants were assayed for phosphorus content using a commercially available kit [Sigma, St. Louis, Mo., USA] (n=3 for each experimental group and time). Beads were then lyophilized, mixed with KBr (˜1 wt %), and made into KBr pellets for Fourier transform infrared spectroscopy (FTIR) analysis as outlined below. In some cases, the cloudy supernatant of the incubation buffer was centrifuged to a pellet, washed with acetone, air dried, and analyzed as a KBr pellet via FTIR.

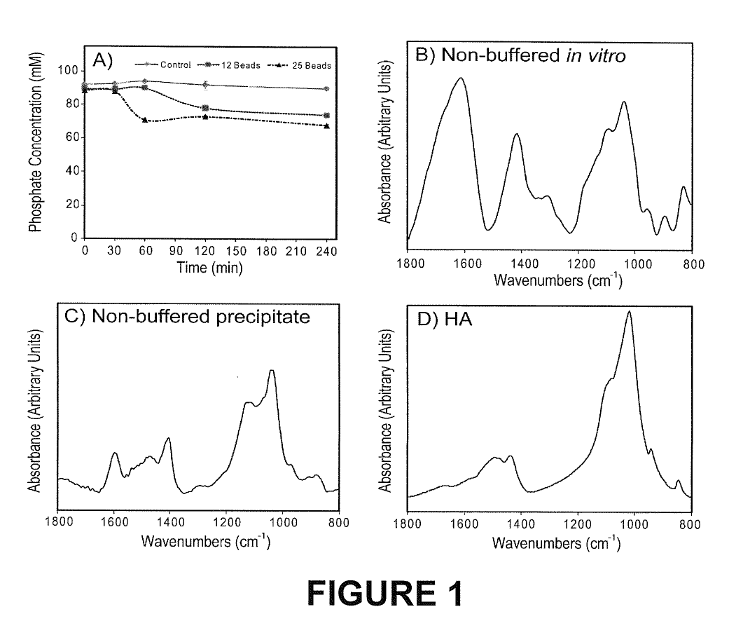

Upon adding the calcium-crosslinked alginate beads to the phosphate buffer, the solution started to become cloudy. The phosphate concentration in the bath decreased by 20-35% over the first 4 hours and depended on the amount of alginate that was added (FIG. 1A). The FTIR spectrum of the lyophilized alginate had peaks between 1600-1800 cm⁻¹, 1370-1525 cm⁻¹, and 900-1200 cm⁻¹ (FIG. 1B) while the FTIR spectrum of the phosphate bath pellet had a similar peak between 900-1200 cm⁻¹ along with peaks centered around 1600 and 1400 cm⁻¹ (FIG. 1C). Pure hydroxyapatite had characteristic peaks between 1400-1550 cm⁻¹ and 900-1200 cm⁻¹ (FIG. 1D).

### Example 2

### Cell Isolation and Culture

Adipose stem cells (ASCs) were isolated. Fat was excised from male and female patients less than 18 years of age undergoing cosmetic and reconstructive procedures at Children's Healthcare of Atlanta under an approved IRB protocol at Georgia Institute of Technology and Children's Healthcare of Atlanta. All patients and parents gave written consent to both the procedure and handling of fat thereafter. ASCs were isolated via a collagenase digestion solution as previously described (Zuk P. A. et al.; Human adipose tissue is a source of multipotent stem cells; Mol Biol Cell. 2002;13:4279-95). Cells were then seeded at 5,000 cells/cm² and cultured in Lonza Mesenchymal Stem Cell Growth Medium [Lonza, Basel, Switzerland] up to second passage.

### Example 3

### Alginate Bead and Microbead Fabrication

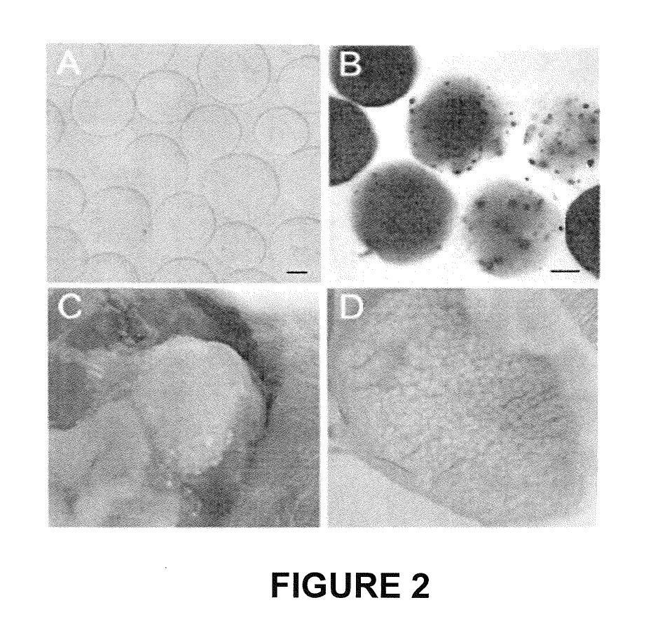

Alginate microbeads were formed in different crosslinking solutions. Medium molecular weight alginate (240,000 kDa) with a high guluronate to mannuronate ratio (69% guluronate) [FMC Biopolymer, Drammen, Norway] was UV light sterilized and dissolved in 155 mM sodium chloride [Ricca Chemical, Arlington, Tex., USA] at a concentration of 20 mg/ml. Alginate containing ASCs was initially seeded at 1×10⁶ cells/ml, resulting in a final measured cell number of 40±7 cells per microbead (FIG. 2B). Microspheres were created using a Nisco Encapsulator VAR V1 LIN-0043 [Nisco Engineering AG, Zurich, Swizterland] at a 4 ml/hr flow rate, 0.175 mm nozzle inner diameter, and 6 kV electrostatic potential. Microbeads were made in four different crosslinking solutions: (i) 50 mM CaCl₂ and 150 mM glucose (non-buffered); (ii) 50 mM CaCl₂ and 150 mM glucose with 25 μM alendronate [Sigma] (bisphosphonate); (iii) 20 mM BaCl₂ and 150 mM glucose (barium); and (iv) 50 mM CaCl₂ and 150 mM glucose with 15 mM 4-(2-hydroxyethyl)-1-piperazineethanesulfonic acid at pH 7.3 [Sigma] (HEPES-buffered). Microbeads made in non-buffered crosslinking solutions were washed and stored in 155 mM sodium chloride (saline) while microbeads made in the HEPES-buffered crosslinking solution were washed and stored in Dulbecco's modified eagle medium (DMEM) [Invitrogen, Carlsbad, Calif., USA] at 37° C. and 5% CO₂ prior to implantation to allow for longer term storage of ASC microbeads in future studies. Following microencapsulation, microbeads were implanted as described below.

### Example 4

### Cell Viability

To determine whether ASCs were viable after microencapsulation in alginate and remained viable after injection delivery, microencapsulated ASCs suspended in DMEM were injected as described below and cultured for 0, 1, and 2 weeks in Lonza Mesenchymal Stem Cell Growth Medium. Microencapsulated ASCs that were not injected were also cultured for comparison (n=6 for each experimental group and time). Viability was measured using fluorescent confocal microscopy using a LIVE/DEAD Viability Kit following the manufacturer's protocol [Invitrogen]. Briefly, samples were incubated for 30 minutes in a PBS solution containing 10 mM CaCl₂, 4 μM ethidium homodimer-1, and 2 μM calcein and imaged with a LSM 510 confocal microscope [Carl Zeiss MicroImaging Inc., Thornwood, N.Y.].

### Example 5

### Animal Surgeries

Male and female athymic nude (Nu/Nu) mice were housed in the vivarium in the Institute for Bioengineering and Bioscience at the Georgia Institute of Technology and handled under a protocol approved by the IACUC committee. Prior to surgeries, athymic mice were anesthetized using isoflurane gas. Both non-buffered and HEPES-buffered microbeads were directly implanted subcutaneously or intramuscularly or injected subcutaneously to determine if delivery method affected alginate calcification. Bisphosphonate and barium microbeads were only injected subcutaneously to reduce animal discomfort and to investigate how the crosslinking solution affects alginate calcification. For intramuscular implants, a small skin incision was made over the calf region of the hind limb, a pouch was prepared in the muscle by blunt dissection, and approximately 0.1 ml microbeads were inserted directly into the gastrocnemius muscle. For all subcutaneous injections, 0.25 ml microbeads were mixed in 0.25 ml DMEM and injected via an 18 gauge needle. Animals were euthanized by CO₂ inhalation at various time points from 1 to 6 months. Each animal received either 2 injections or 2 implantations subcutaneously or 2 bilateral intramuscular implantations (n=4-6 for each experimental condition). Samples were harvested and processed for subsequent studies as described below.

### Example 6

### Micro-Computed Tomography

To assess the extent of alginate microbead mineralization, subcutaneous and intramuscular samples were excised from nude mice, immediately scanned using a μJET 40 (Scanco Medical, Switzerland) with a voxel size of 20 μm, and analyzed as previously described (Boyan B. D. et al.;Regulation of growth plate chondrocytes by 1,25-dihydroxyvitamin D3 requires caveolae and caveolin-1; J Bone Miner Res. 2006;21:1637-47). Calcification was identified using a fixed threshold; individual samples were isolated with user-guided contours and three dimensional images were created. Samples were then fixed in 10% neutral buffered formalin [Sigma] for histological processing or frozen and lyophilized for subsequent materials characterization.

### Example 7

### Histology

After 48 hours of fixation in formalin, representative undecalcified samples were embedded in plastic, and cut into 10-μm thick sections. Samples were stained with von Kossa with a nuclear fast red counter stain as previously described (Rubin J. et al.; Caveolin-1 knockout mice have increased bone size and stiffness; J Bone Miner Res. 2007;22:1408-18).

Microbeads formed in all crosslinking solutions ranged from 200-350 μm in diameter (FIG. 2A). When non-buffered microbeads were either implanted or injected subcutaneously into male nude mice, almost every sample showed the presence of mineral at all time points examined (1, 3, and 6 months; Table 1). Specifically, 24 of 24 implanted samples calcified whereas 21 of 24 injected samples calcified. ASC viability prior to implantation or injection was 70±3% (FIG. 2B), and in vitro studies show cell viability increasing to 80±5% two weeks post injection (data not shown), yet the presence of ASCs had no apparent effect on mineralization (Table 1). Mineralization was demonstrated by light microscopy (FIG. 2C) or by visual inspection of implanted (FIG. 2D) and injected (FIG. 2E) microbeads.

TABLE 1
Mineralization of cellular and acellular microbeads in male
nude mice with different delivery methods at
different times subcutaneously based on visual inspection.

| Months Post | | |
|---|---|---|
| Op | Empty | ASC-seeded |
| 1 | Implantation: 4/4 Mineralized | Implantation: 4/4 Mineralized |
| | Injection: 3/4 Mineralized¹ | Injection: 3/4 Mineralized |
| 3 | Implantation: 4/4 Mineralized | Implantation: 4/4 Mineralized |
| | Injection: 3/4 Mineralized¹ | Injection: 4/4 Mineralized |
| 6 | Implantation: 4/4 Mineralized | Implantation: 4/4 Mineralized |
| | Injection: 4/4 Mineralized | Injection: 4/4 Mineralized |

¹Microbeads for one sample disappeared and had no volume retention

Modifications to the crosslinking protocol reduced or eliminated calcification as detected by von Kossa staining (Table 2). When microbeads were injected subcutaneously, no visual calcification was evident when barium chloride was used as the crosslinker and the addition of the 25 μM bisphosphonate to the crosslinking solution partially reduced mineralization. HEPES-buffered (pH 7.3) microbeads injected subcutaneously also had no apparent calcification. When HEPES-buffered samples were then directly implanted subcutaneously and intramuscularly, there was no visual mineralization. In contrast, when non-buffered microbeads were implanted a second time, all the subcutaneous and two-thirds of the intramuscular samples mineralized.

TABLE 2
Attempts to regulating calcification by modifying the crosslinking
solution and delivery location 5-8 weeks
post implantation or injection based on von Kossa staining.

| | | | Bisphos- | HEPES- |
|---|---|---|---|---|
| | Non-buffered | Barium | phonate | Buffered |
| Implantation | Subcutaneous: | — | — | Subcutaneous: |
| | 4/4 Calcified | | | 0/4 Calcified |
| | Intramuscular: | — | — | Intramuscular: |
| | 4/6 Calcified | | | 0/4 Calcified |
| Injection | — | 0/4 Calcified | 2/4 Calcified | 0/4 Calcified |

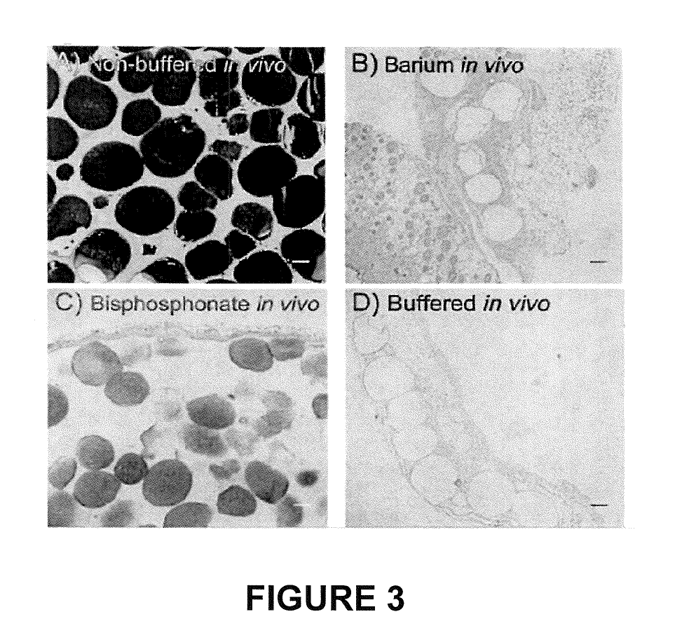

The intensity of von Kossa staining for phosphate was very strong in non-buffered samples as phosphate was present throughout almost every microbead (FIG. 3A). Barium chloride-treated samples had no detectable presence of von Kossa staining with microbeads surrounded by endothelial tissue (FIG. 3B). Bisphosphonate-treated samples that did calcify only had partially positive staining for phosphate as both the intensity of staining and the number of positively stained microbeads was lower compared to non-buffered samples (FIG. 3C, Table 2). HEPES-buffered samples had no von Kossa staining and were surrounded by connective tissue (FIG. 3D).

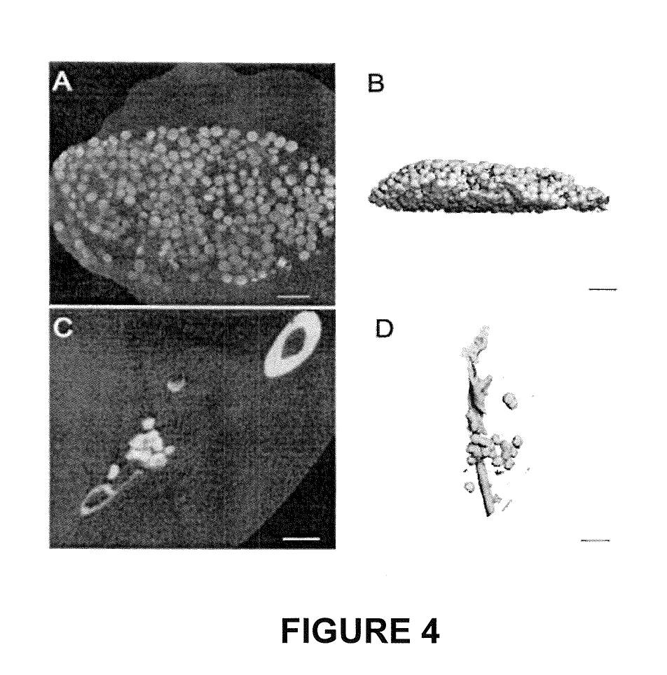

Cross-sectional x-ray sections of non-buffered samples via microCT showed extensive mineralization that was not just limited to the surfaces of individual microbeads or peripheral microbeads of the bolus (FIGS. 4A, B). Additionally, the intensity of X-ray attenuation seemed to be comparable to the adjacent bone. 3-D reconstructions further demonstrate the extent of calcification of both subcutaneous (FIG. 4C) and intramuscular (FIG. 4D) samples. HEPES-buffered samples were undetectable by microCT (data not shown).

### Example 8

### Fourier Transform-Infrared Spectroscopy (FTIR)

To test our hypothesis that alginate calcification mineral was similar to hydroxyapatite found in bone, infrared spectroscopy in attenuated total internal reflection (ATR) mode [Pike Technologies, Madison, Wis., USA] was performed on lyophilized samples using a Nexus 870 FT-IR bench [Nicolet Instrument Corporation, Madison, Wis., USA]. Each spectrum was the mean of two acquisitions (between 1800 and 800 cm−1) of at least 64 scans with a spectral resolution of 4 cm⁻¹.

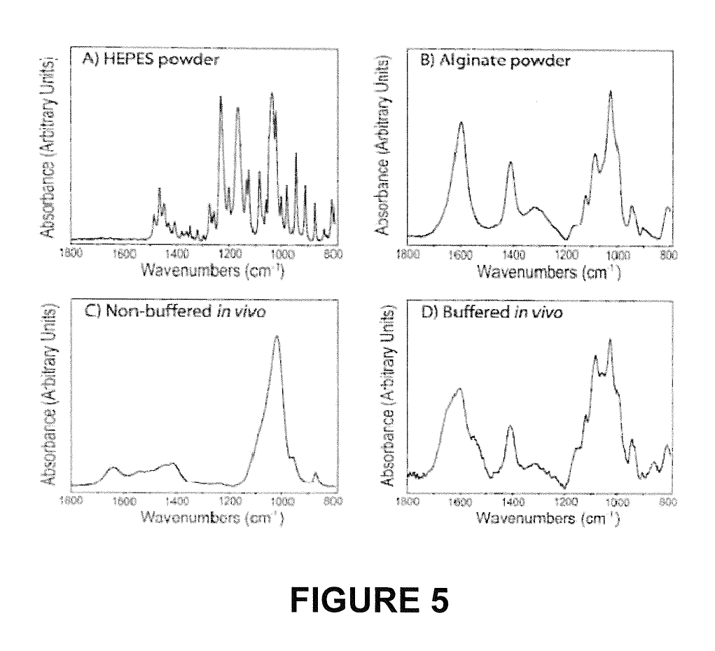

Comparison of FTIR spectra of the lyophilized explanted samples presented significant differences. The spectrum of the non-buffered sample (FIG. 5A) showed the characteristic bands of hydroxyapatite around 1400-1550 cm⁻¹ and 900-1200 cm⁻¹ and corresponded well with the spectrum of the pure hydroxyapatite powder. The spectrum of the HEPES-buffered sample (FIG. 5B) had no traces of the HEPES spectrum (FIG. 5C), but matched almost perfectly with the spectrum of the pure alginate powder (FIG. 5D). No presence of hydroxyapatite was noted in the HEPES-buffered sample.

### Example 9

### X-Ray Diffraction

Crystal structure of the samples was identified using an)(Pert PRO Alpha-1 diffractometer [PANalytical, Almelo, The Netherlands]. X-ray diffraction (XRD) scans were collected using Cu Kα radiation. A 1° parallel plate collimator, ½ divergence slit and 0.04 rad soller slit were used for controlled axial divergence. Bragg-Brentano parafocusing at 45 kV and 40 mA was used to analyze samples. The assignment of detected peaks to crystalline phases was performed using the database from the International Centre for Diffraction Data (ICDD, 2008).

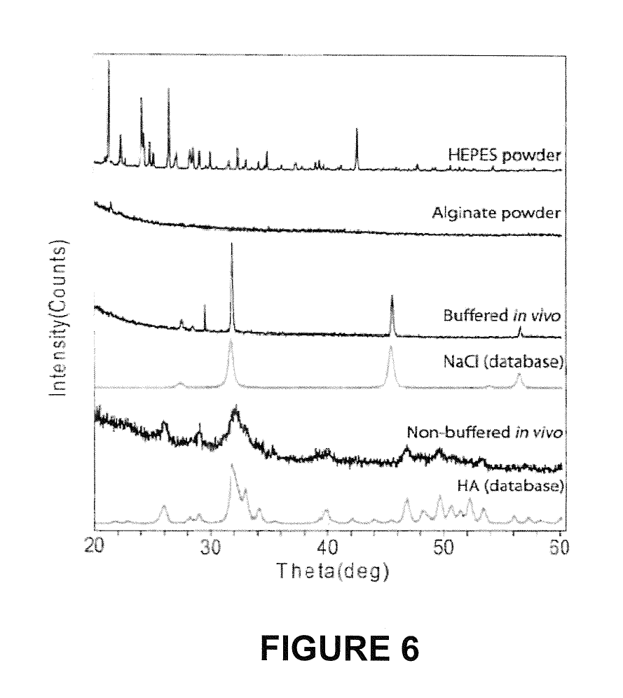

The XRD spectra of the explanted samples showed the presence of monphasic crystalline structures (FIG. 6). The alginate powder presented no diffraction pattern. The non-buffered sample appeared to have the crystal structure of hydroxyapatite, whereas the HEPES-buffered sample showed the main peaks of NaCl crystals and incorporated none of the peaks of the HEPES buffer. The crystalline structures were further confirmed with the EDS spectra (Table 3), which showed the presence of 10.2±1.3% Ca and 6.3±0.8% P on the non-buffered sample (Ca/P ratio of 1.6±0.4), and only approximately 1% of each on the HEPES-buffered sample. Conversely, the HEPES-buffered sample included 14.3±0.7% Na and 13.2±1.8% Cl, and the non-buffered sample had <1% of Na and no traces of Cl. The main components of both samples were C and O, primarily from the alginate polymer.

TABLE 3
EDS calculated elemental composition of non-buffered and buffered in vivo samples

| | Concentration [atomic %]^{1,2} | | | | | | | | | |
|---|---|---|---|---|---|---|---|---|---|---|
| | C | O | Na | Mg | Al | P | S | Cl | Ca | K |
| Non- | 34.0 ± 4.3 | 47.2 ± 2.0 | <1 | <1 | 1.2 ± 0.4 | 6.3 ± 0.8 | <1 | — | 10.2 ± 1.3 | — |
| buffered | | | | | | | | | | |
| HEPES- | 41.6 ± 1.0 | 27.5 ± 1.9 | 14.3 ± 0.7 | — | <1 | 1.15 ± 0.1 | <1 | 13.2 ± 1.8 | <1 | <1 |
| Buffered | | | | | | | | | | |

¹The values should be evaluated with an error of approximately ±2% relative.
²Elements that were not present in all measurements of the same sample were not included in the table (e.g., Si).

Investigation using FTIR, XRD, and EDS showed that hydroxyapatite is the most stable crystal phase formed when alginate is calcified. Explanted non-buffered alginate microbeads had similar spectra to pure hydroxyapatite for both FTIR and XRD whereas buffered microbeads appeared to be a combination of alginate powder and salt crystals when both analytical modalities were used. Specifically, FTIR spectrum of non-buffered microbeads closely resembled that of 16-day-old rat calvaria with characteristic phosphate (900-1180 cm⁻¹) and amide I (1580-1750 cm⁻¹) peaks. Although, the broad phosphate peak in the non-buffered microbeads does overlap with aryl-hydroxyl (1030-1085 cm⁻¹) and carboxylic acid (915-995 cm⁻¹) groups found in the HEPES-buffered microbeads and alginate powder, the disappearance of alginate peaks found at lower frequencies, the low intensities of the amide I and II peaks (1405-1420, 1600-1690 cm⁻¹) in the non-buffered samples, and the XRD spectra suggest the formation of hydroxyapatite. To confirm these findings, the Ca/P ratio for non-buffered alginate was found to be 1.6±0.4, which closely matches hydroxyapatite found in bone. Non-buffered microbeads also had traces of Mg, which has been associated with facilitating the formation of calcified pathological cardiovascular deposits. HEPES-buffered microbeads only had traces of calcium left, suggesting that these samples were starting to be reabsorbed. The presence of sulfur and higher content of carbon in HEPES-buffered microbeads compared to non-buffered samples suggest levels of tissue incorporation, which was also confirmed with histology.

### Example 10

### Scanning Electron Microscopy and Energy Dispersive X-Ray Spectroscopy

Morphology of the microbeads was qualitatively evaluated using an Ultra 60 field emission scanning electron microscope (FESEM) [Carl Zeiss SMT Ltd., Cambridge, UK] at an accelerating voltage of 5 kV and different magnifications. Chemical composition of samples was determined using an INCAPentaFET-x3 energy dispersive x-ray spectrometer (EDS) [Oxford Instruments, Bucks, UK] at an accelerating voltage of 15 kV and a working distance of 8.5 mm.

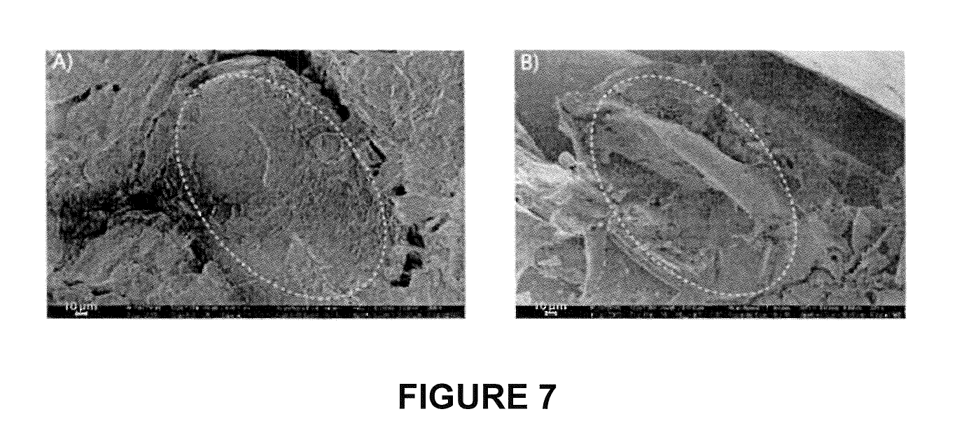

SEM image of non-buffered microbeads that were lyophilized shows an intact spherical structure with surrounding tissue growth (FIG. 7A) whereas HEPES-buffered microbeads that were lyophilized were clearly fragmented (FIG. 7B).

As shown above, alginate microbead calcification did not strongly depend on the delivery method, delivery site, the presence of cells, or sex of the animal, although in other studies, it has been shown that biological factors can play a significant role in mineralizing alginate constructs in vivo.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method of inhibiting or preventing calcification of a hydrogel in vivo comprising:
combining the hydrogel with a non-phosphate buffer solution having a pH of less than 7.4; and
administering the hydrogel to a subject,
wherein the hydrogel is formed by crosslinking a polyanionic polymer with a polycation.

2. The method of claim 1, wherein combining the hydrogel with the non-phosphate buffer solution comprises:
crosslinking the polyanionic polymer with the polyvalent cation in the non-phosphate buffer solution to form the hydrogel.

3. The method of claim 1, wherein the polyanionic polymer comprises a polyanionic polysaccharide.

4. The method of claim 3, wherein the polyanionic polysaccharide comprises alginate and the hydrogel is alginate hydrogel.

5. The method of claim 1, wherein the hydrogel is administered as particles having a diameter in a range of 30 μm to 2 mm.

6. The method of claim 5, wherein the particles have a diameter in a range of 175 μm to 350 μm.

7. The method of claim 1, wherein the hydrogel encapsulates biological material.

8. The method of claim 7, wherein the biological material is encapsulated during crosslinking of the hydrogel.

9. The method of claim 1, wherein the non-phosphate buffer solution comprises 4-(2-hydroxyethyl)-1-piperazineethanesulfonic acid (HEPES).

10. The method of claim 1, wherein the non-phosphate buffer solution has a pH of less than 7.3.

11. The method of claim 1, wherein administering the hydrogel comprises injecting and/or implanting the hydrogel into the subject.

12. The method of claim 1, wherein the hydrogel does not calcify in the subject within 8 weeks.

13. A method of inhibiting or preventing calcification of a hydrogel in vivo comprising:
forming a hydrogel by crosslinking a polyanionic polymer with a polycation in a solution comprising a bisphosphonate compound; and
administering the formed hydrogel to a subject.

14. The method of claim 13, wherein the polyanionic polymer comprises a polyanionic polysaccharide.

15. The method of claim 14, wherein the polyanionic polysaccharide comprises alginate and the hydrogel is alginate hydrogel.

16. The method of claim 13, wherein the hydrogel is administered as particles having a diameter in a range of 30 μm to 2 mm.

17. The method of claim 16, wherein the particles have a diameter in a range of 175 μm to 350 μm.

18. The method of claim 13, wherein the hydrogel encapsulates biological material.

19. The method of claim 18, wherein the biological material is encapsulated during crosslinking of the hydrogel.

20. The method of claim 13, wherein the bisphosphonate compound comprises aledronic acid and/or a salt thereof.

21. The method of claim 13, wherein the bisphosphonate compound is present in the solution at a concentration in a range of 1 μM to 1 mM.

22. The method of claim 13, wherein administering the hydrogel comprises injecting and/or implanting the hydrogel into the subject.

23. The method of claim 13, wherein the hydrogel does not calcify in the subject within 8 weeks.

24-40. (canceled)

41. A hydrogel composition comprising:
hydrogel formed by crosslinking a polyanionic polymer and a polycation; and
a bisphosphonate compound.

42. The composition of claim 41, wherein the hydrogel comprises polyanionic polysaccharide.

43. The composition of claim 42, wherein the hydrogel is alginate hydrogel.

44. The composition of claim 41, wherein the bisphosphonate compound comprises aledronic acid and/or a salt thereof.

45. The composition of claim 41, wherein the bisphosphonate compound and hydrogel are present in a solution, and
the bisphosphonate compound is present in the solution at a concentration in a range of 1 μM to 1 mM.

46-49. (canceled)
